(19) United States
(12) Patent Application Publication
(10) Pub. No.: US 2016/0067170 A1
(43) Pub. Date: Mar. 10, 2016

(54) ORAL AND DENTAL HYGIENE AND CLEANING AGENTS WITH IMPROVED ANTI-BACTERIAL ACTION

(71) Applicant: Henkel AG & Co. KGaA, Duesseldorf (DE)

(72) Inventors: Thomas Welss, Duesseldorf (DE); Thomas Foerster, Duesseldorf (DE); Markus Semrau, Langenfeld (DE); Kristin Miehlich, Wuppertal (DE); Claudia Hundeiker, Meerbusch (DE)

(21) Appl. No.: 14/944,924

(22) Filed: Nov. 18, 2015

Related U.S. Application Data
(63) Continuation of application No. PCT/EP2014/052039, filed on Feb. 3, 2014.

(30) Foreign Application Priority Data
May 28, 2013 (DE) 10 2013 209 892.8

(51) Int. Cl. A61K 8/85; A61K 8/23; A61K 8/34; A61K 9/16; A61K 8/49; A61Q 11/00; A61K 31/765; A61K 33/38; A61K 31/194; A61K 31/235; A61K 33/34; A61K 31/085; A61K 31/505; A61K 9/00; A61K 8/365

(52) U.S. Cl. CPC A61K 8/85; A61K 8/365; A61K 8/23; A61K 8/347; A61K 9/16; A61K 8/4953; A61Q 11/00; A61K 31/765; A61K 33/38; A61K 31/194; A61K 31/235; A61K 33/34; A61K 31/085; A61K 31/505; A61K 9/0053; A61K 2800/412; A61K 2800/28; A61K 2800/596; A61K 2800/92

(57) ABSTRACT

The anti-inflammatory and preventive action of antimicrobial substances can surprisingly be enhanced by the use of polylactic acid particles. Corresponding advantageous products are in particular oral and dental care and cleaning products that—based on the weight thereof—comprise 0.001 to 25 wt. % polylactic acid particles and 0.00001 to 5 wt. % of at least one antibacterial compound from the groups consisting of the benzoates and/or the parabens and/or the Cu salts and/or the Ag salts and/or triclosan and/or hexetidine.

### FIELD OF THE INVENTION

The present invention generally relates to preparations for oral and dental care and cleaning which due to special ingredients have excellent gum care and gum-preserving action.

### BACKGROUND OF THE INVENTION

Dental cleaning products are available on the market in various forms and are used primarily to clean the surface of the teeth and prevent tooth and periodontal diseases. They usually include a combination of polishing agents, humectants, surfactants, binding agents, flavoring agents, and fluoride-containing and antimicrobial active substances. In addition to tooth powders, which due to the increased abrasiveness thereof play a lesser role, dental cleaning products are offered above all in paste, cream and translucent or transparent gel forms. In recent years, liquid toothpastes and mouthwashes have also increasingly gained in importance.

In addition to cleaning of the teeth, the consumer also expects products of the type in question to provide care of the teeth and of the oral cavity. For example, in particular a “clean” sensation, which is to say a smooth and shiny tooth surface and a fresh sensation in the mouth, are key aspects in terms of the incentive to purchase preparations for oral and dental care and cleaning. A successful product of the type in question should therefore thoroughly clean the teeth without damaging the tooth or the tooth surface, while also reducing and/or preventing bad breath.

Bad breath, also referred to as halitosis or “foetor ex ore,” can have a variety of causes. In healthy individuals, however, bad breath in most instances is caused by bacteria in the oral cavity or pharyngeal space, which metabolize body cells or food residue, wherein volatile sulfur compounds (VSCs), among other things, result from the breakdown of proteins and are partly responsible for the malodor.

These sulfur compounds are in particular: H₂S=hydrogen sulfide (approximately 30%), CH₃—S—H=methyl mercaptan (approximately 60%), and CH₃—S—CH₃=dimethyl sulfide (approximately 10%).

Some of the volatile sulfur compounds are very aggressive and may damage the gingiva and the oral mucosa. It is known, for example, that hydrogen sulfide and methyl mercaptan increase the permeability of the oral mucosa and thereby can abet periodontal diseases.

Periodontal diseases pose a high health risk factor. According to studies conducted by the World Health Organization (WHO) more than 50% of adult Germans suffer from periodontopathies in urgent need of treatment. Periodontitis is understood to mean an inflammatory change of the tissue surrounding the tooth, in particular of the jaw bone, caused by bacteria. Periodontitis (=involving the gingival pockets and the bone) always develops from gingivitis and initially affects the interdental spaces most difficult to access for cleaning. The bacteria present in the gingival pocket act destructively on the gingiva, tooth substance and bone through their metabolic products, in particular through the VSCs produced by them, and thus continually intensify any existing periodontitis.

Many attempts have been made to combat bad breath and inflammatory conditions in the oral cavity, and a plurality of agents have been used for this purpose in preparations for oral and dental care and cleaning. For example, triclosan, zinc salts or chlorhexidine are used as antibacterial agents. However, the use of these products may be associated with disadvantages in isolated cases, which in some instances are related to taste, in others have aesthetic reasons, since the product and/or the teeth may become discolored from excessive use of the agents. In isolated cases, fighting the bacteria may not be sufficient to achieve a drop in the inflammatory state.

As a result, there continues to be a need to fight halitosis and to provide active substances or active substance combinations that are free from the described disadvantages.

The use of polylactic acid particles in oral hygiene products is described in the international patent applications WO2012/177616 and WO2012/177617. There, however, the particles are disclosed exclusively as biodegradable abrasives.

It was the object of the present invention to provide preparations for oral and dental care and cleaning which effectively combat bad breath and are effective against gingivitis and periodontitis. In one aspect of the invention, it was to be possible to avoid the disadvantages of the use of triclosan, zinc salts or chlorhexidine, reduce the amounts used or draw on natural anti-microbial substances. In a further aspect of the invention, the care of the oral cavity was to be improved by supporting the natural regeneration of the oral mucosa and of the gingiva.

It has now been found that polylactic acid particles are suitable for enhancing the antibacterial action of certain compounds and for solving the above-described set of problems.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An oral and dental care and cleaning product, comprising—based on the weight thereof—0.001 to 25 wt. % polylactic acid particles, and 0.00001 to 5 wt. % of at least one antibacterial compound from the groups consisting of the benzoates, and/or of the parabens, and/or of the Cu salts, and/or of the Ag salts, and/or triclosan, and/or hexetidine.

Use of polylactic acid particles to enhance the action of antibacterial compounds.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In a first embodiment, the subject matter of the present invention is an oral and dental care and cleaning product, comprising—based on the weight thereof—

a) 0.001 to 25 wt. % polylactic acid particles, and

b) 0.00001 to 5 wt. % of at least one antibacterial compound from the groups consisting
a. of the benzoates and/or
b. of the parabens and/or
c. of the Cu salts and/or
d. of the Ag salts and/or
e. triclosan and/or
f. hexetidine.

Oral and dental care products and oral and dental cleaning products within the meaning of the invention are oral and tooth powders, oral pastes and toothpastes, liquid oral creams and dentifrices, oral and dental rinses, and oral and dental gels. Toothpastes and liquid dental cleaning products are preferably suited. For this purpose, the oral and dental care and cleaning products may be present, for example, in the form of toothpastes, liquid dentifrices, tooth powders, mouthwashes or optionally also as a chewing compound, such as chewing gum. However, they are preferably present as substantially flow-able or plastic toothpastes, as they are used to clean teeth along with a toothbrush. A further particularly preferred embodiment of the present invention is oral rinse solutions and mouthwashes that are used to rinse out the oral cavity.

The first essential ingredient present in the compositions according to the invention is 0.001 to 25 wt. % polylactic acid particles, based on the weight of the compositions.

Preferred products according to the invention use the polylactic acid particles within narrower quantity ranges. Oral and dental care and cleaning products are preferred here which—based on the weight thereof—comprise 0.002 to 20 wt. %, preferably 0.003 to 17.5 wt. %, particularly preferably 0.004 to 15 wt. %, exceptionally preferably 0.005 to 12.5 wt. %, and in particular 0.01 to 10 wt. % polylactic acid particles.

Polylactic acid, also referred to as polylactide or PLA, is a designation for biodegradable polymers (polyesters) which are obtainable primarily by the ionic polymerization of lactide, a cyclic compound of two lactic acid molecules.

Ring-opening polymerization takes place at temperatures between 140 and 180° C. and under the action of catalytic tin compounds (such as tin oxide). In this way, plastic materials having high molecular masses and strength are created. Lactide itself may be prepared by fermentation of molasses or by fermentation of glucose using various bacteria.

In addition, high molecular weight and pure polylactides may be produced directly from lactic acid using what is known as polycondensation. In industrial production, however, disposal of the solvent poses a problem.

Lactic acid (2-hydroxypropanoic acid) has an asymmetric carbon atom, so that polylactic acid also has optically active centers with L(+) and D(−) configuration. The ratio of L to D monomeric units determines the degree of crystallinity, the melting point, and the biodegradability of the polymers.

Suitable polylactic acids according to the invention are L-polylactic acid, D-polylactic acid, and L,D-polylactic acid and the mixtures thereof. L-polylactic acid is particularly preferred due to the excellent biodegradability thereof. In a preferred embodiment of the present invention, the weight fraction of L-lactic acid monomeric units in the polylactic acid is greater than 50 wt. %, preferably greater than 80 wt. %, and in particular greater than 90 wt. %.

The molar mass of polylactic acid is usually 1000 to 1,000,000, preferably 10,000 to 300,000, more preferably 50,000 to 250,000, and in particular 100,000 to 180,000 dalton.

In a further preferred embodiment of the present invention, the polylactic acid is used in a form that is blended with fillers. The use of larger filler amounts helps comminute the polymer into particles and increases biodegradability and the inner specific surface area via porosity and capillarity. In particular water-soluble fillers are preferred here, for example metal chlorides such as NaCl, KCl and the like, metal carbonates such as Na₂CO₃, NaHCO₃ and the like, and metal sulfates such as MgSO₄.

It is also possible to use natural raw materials as fillers, such as nut shells, wood or bamboo fibers, starch, xanthan, alginates, dextran, agar and the like. These fillers are biodegradable and do not worsen the good ecological properties of the polylactic acid particles. The content of biodegradable fillers in the polylactic acid particles can usually be 10 to 70 wt. %, wherein quantities from 20 to 60 are preferred, and those from 30 to 50 wt. % are particularly preferred.

While the polylactic acid particles are used within the scope of the present invention not because of the abrasive properties thereof, irregular shapes have proven to be particularly preferred since this allows the action according to the invention to be increased yet again compared to spherical particles.

Particularly suited particles according to the invention have a circularity between 0.1 and 0.6.

The shape of the polylactic acid particles used according to the invention can be defined in various way, wherein the geometric proportions of a particle and—more pragmatically—of a particle aggregate are determined within the scope of this preferred embodiment of the present invention.

Newer high-precision methods allow particle shapes to be precisely determined from a large number of particles, usually more than 10,000 particles, preferably more than 100,000 particles. These methods make it possible to precisely select the average particle shape of a particle aggregate. The particle shapes are preferably determined by way of an “Occhio Nano 500 Particle Characterisation Instrument” using the software “Callistro version 25” (Occhio s.a. Liege, Belgium). This device allows the preparation, dispersion, imaging and analysis of a particle aggregate, wherein preferably the device parameters are set as follows: White Requested=180, vacuum time=5000 ms, sedimentation time=5000 ms, automatic threshold, number of particles counted/analyses=8000 to 500000, minimum number of replicates/sample=3, lens setting 1×/1.5×.

The polylactic acid particles used according to the invention preferably have sizes that are characterized by the area equivalent diameter thereof (ISO 9276-6:2008(E) section 7), also referred to as “equivalent circle diameter, ECD” (ASTM F1877-05 Section 11.3.2). The mean ECD of a particle aggregate is calculated as the average ECD of each individual particle of a particle aggregate of at least 10,000 particles, preferably of more than 50,000 particles, in particular of more than 100,000 particles, after particles having an area equivalent diameter (ECD) of less than 10 μm were excluded from the measurement.

In a preferred embodiment of the present invention, the polylactic acid particles have mean ECD values of 10 to 1000 μm, preferably of 50 to 500 μm, more preferably of 100 to 350 μm, and in particular of 150 to 250 μm.

Regardless of the mean particle size, oral and dental care and cleaning products according to the invention are preferred in which the polylactic acid particles have absolute particle sizes of 1 to 1000 μm, preferably of 2 to 750 μm, and in particular of 10 to 500 μm.

Within the scope of the present invention, shape descriptors are used which are calculations from geometric descriptors or form factors. Form factors are ratios between two different geometric properties, which in turn are a measurement of the proportions of the image of an entire particle or the measurement of the proportions of an ideal geometric body that surrounds the particles.

These results are descriptors which are similar to aspect ratios. In a preferred embodiment of the present invention, meso-form descriptors are used for particle characterization. These meso-form descriptors indicate the extent to which a particle deviates from an ideal geometric shape, in particular from a sphere. In the preferred embodiment of the present invention, the polylactic acid particles differ from the typical spherical shape or sphere-like shape, such as granular particles.

The particles preferably have sharp corners and edges and preferably have concave indentations. Sharp edges of non-spherical particles can be defined by a radius of less than 20 μm, preferably less than 8 μm, and in particular less than 5 μm, wherein the radius is defined as the radius of an imaginary circle that follows the contour of the corner.

Circularity is a quantitative, two-dimensional image analysis and can be determined according to ISO 9276-6:2008(E) section 8.2. Circularity is a preferred meso-form descriptor and can be determined, for example, by way of the above-described “Occhio Nano 500 Particle Characterisation Instrument” using the software “Callistro version 25” (Occhio s.a. Liege, Belgium) or by way of the “Malvern Morphologi G3.” Circularity is on occasion described in the literature as the difference between a particle and the perfect spherical shape. The values for circularity vary between 0 and 1, with 1 describing the perfect sphere or (in the two-dimensional image) the perfect circle: C=[(4πA)/P²]^{1/2}

where A denotes the projected area (the two-dimensional descriptor) and P denotes the length of the perimeter of the particle.

Polylactic acid particles having a mean circularity C of 0.1 to 0.6, preferably of 0.15 to 0.4, and in particular of 0.2 to 0.35 have proven to be particularly suited within the scope of the present invention. The mean values can be found by forming quotients from volume-based measurements and number-based measurements.

Solidity is a quantitative, two-dimensional image analysis and can be determined according to ISO 9276-6:2008(E) section 8.2. Solidity is likewise a preferred meso-form descriptor and can be determined, for example, by way of the above-described “Occhio Nano 500 Particle Characterisation Instrument” using the software “Callistro version 25” (Occhio s.a. Liege, Belgium) or by way of the “Malvern Morphologi G3.” Solidity is a meso-form descriptor that describes the concavity of a particle or of a particle aggregate. The values for solidity vary between 0 and 1, with 1 describing a non-concave particle: solidity=A/Ac

where A describes the (image) area of the particle and Ac describes the convex hull area surrounding the particle.

Polylactic acid particles having a mean solidity of 0.4 to 0.9, preferably of 0.5 to 0.8, and in particular of 0.55 to 0.65, have proven to be particularly suited within the scope of the present invention. The mean values can be found by forming quotients from volume-based measurements and number-based measurements.

In particularly preferred embodiments of the present invention, the polylactic acid particles used have a mean circularity C of 0.1 to 0.6, preferably of 0.15 to 0.4, and in particular of 0.2 to 0.35, and a mean solidity of 0.4 to 0.9, preferably of 0.5 to 0.8, and in particular of 0.55 to 0.65.

The “mean” circularities and solidities are average values from the measurement of a large number of particles, usually more than 10,000 particles, preferably more than 50,000 particles, and in particular more than 100,000 particles, wherein particles having an area equivalent diameter (ECD) of less than 10 μm were excluded from the measurement.

After the polylactic acid polymer is produced, the polymer can be brought into the desired particle size and shape, for example by grinding methods.

A particularly preferred method for producing the particles having the desired circularity and solidity is that of producing a foam made of polylactic acid, followed by pulverizing. While the polylactic acid particles are used within the scope of the present invention not because of the abrasive properties thereof, polylactic acid particles having a certain hardness have proven to be particularly preferred since this allows the action according to the invention to be enhanced yet again compared to particles that are too hard or too soft.

The hardness of the particles can be varied via the ratio of D to L monomers and via the molar mass.

Preferred polylactic acid particles have hardnesses of 3 to 50 kg/mm², preferably of 4 to 25 kg/mm², and in particular of 5 to 15 kg/mm₂ on the HV, Vickers hardness scale.

A preferred group of anti-microbial compounds according to the invention, which can be used in the products according to the invention in addition to the polylactic acid particles, is benzoates. For example, benzoic acid (E 210), sodium benzoate (E 211), potassium benzoate (E 212), calcium benzoate (E 213), ethyl 4-hydroxybenzoate (E 214), ethyl 4-hydroxybenzoate, sodium salt (E 215), propyl 4-hydroxybenzoate (E 216), propyl 4-hydroxybenzoate, sodium salt (E 217), methyl 4-hydroxybenzoate (E 218) or methyl 4-hydroxybenzoate, sodium salt (E 219) can be used in oral and dental care and cleaning products. The use of sodium benzoate and/or esters of p-hydroxybenzoic acid is most particularly preferred, wherein particularly preferred oral and dental care and cleaning products according to the invention are characterized by comprising—based on the weight thereof—0.01 to 4 wt. %, preferably 0.02 to 3 wt. %, particularly preferably 0.05 to 2 wt. %, exceptionally preferably 0.1 to 1.5 wt. %, and in particular 0.1 to 1.0 wt. % p-hydroxybenzoic acid methyl-, -ethyl- or -propyl ester or the mixtures thereof.

Likewise preferred oral and dental care and cleaning products according to the invention comprise—based on the weight thereof—0.01 to 4 wt. %, preferably 0.02 to 3 wt. %, particularly preferably 0.05 to 2 wt. %, exceptionally preferably 0.1 to 1.5 wt. %, and in particular 0.1 to 1.0 wt. % sodium benzoate.

A further preferred group of anti-microbial compounds according to the invention, which can be used in the products according to the invention in addition to or instead of benzoates, is parabens. Preferred oral and dental care and cleaning products according to the invention comprise—based on the weight thereof—0.01 to 4 wt. %, preferably 0.02 to 3 wt. %, particularly preferably 0.05 to 2 wt. %, exceptionally preferably 0.1 to 1.5 wt. %, and in particular 0.1 to 1.0 wt. % parabens, wherein particularly preferred oral and dental care and cleaning products according to the invention are characterized by comprising—based on the weight thereof—0.01 to 4 wt. %, preferably 0.02 to 3 wt. %, particularly preferably 0.05 to 2 wt. %, exceptionally preferably 0.1 to 1.5 wt. %, and in particular 0.1 to 1.0 wt. % parabens from the group methyl paraben, ethyl paraben and the mixtures thereof.

A further preferred group of anti-microbial compounds according to the invention, which can be used in the products according to the invention in addition to or instead of benzoates and/or parabens, is Cu salts.

In principle, all toxicologically unobjectionable and mucosa-friendly copper compounds are suitable as copper ion-supplying compounds. The following shall be mentioned by way of example of the inorganic salts: copper chloride, CuCl₂ and the dihydrate thereof; copper fluoride, CuF₂, and the dihydrate thereof; copper fluosilicate, CuSiF₆, and the hexahydrate thereof; copper sulfate, CuSO₄, and the pentahydrate thereof; copper nitrate and the trihydrate and hexahydrate thereof, and less common copper salts such as bromides, bromates, chlorates, iodates and fluorophosphates. Preferred copper salts of organic acids are acetate, formiate, benzoate, citrate, tartrate, lactate, malate, mandelate, sorbate, pantothenate, gluconate, phytate, glycerophosphate, cinnamate, butyrate, propionate, laurate, oxalate, and salicylate. The copper salts of amino acids, such as glycinate or glutamate, are also suitable. Copper aspartate is a particularly preferred amino acid salt.

Regardless of the type of copper salts, these are preferably used in quantities such that a certain copper ion concentration in the product is ensured. Preferred oral and dental care and cleaning products according to the invention comprise—based on the weight thereof—0.00001 to 0.1 wt. %, preferably 0.00002 to 0.05 wt. %, particularly preferably 0.00005 to 0.025 wt. %, exceptionally preferably 0.000075 to 0.015 wt. %, and in particular 0.0001 to 0.001 wt. % copper ions, calculated as Cu²⁺.

A further preferred group of anti-microbial compounds according to the invention, which can be used in the products according to the invention in addition to or instead of benzoates and/or parabens and/or Cu salts, is Ag salts. For example, suitable silver salts are silver nitrate, silver acetate, silver lactate, silver phosphate, silver chloride, silver bromide, silver hydroxide, silver carbonate, silver oxide, silver periodate or the sodium chloride/silver chloride complex (Na[AgCl₂]). Regardless of the type of silver salts, these are preferably used in quantities such that a certain silver ion concentration in the product is ensured. Preferred oral and dental care and cleaning products according to the invention comprise—based on the weight thereof—0.00001 to 0.1 wt. %, preferably 0.00002 to 0.05 wt. %, particularly preferably 0.00005 to 0.025 wt. %, exceptionally preferably 0.000075 to 0.015 wt. %, and in particular 0.0001 to 0.001 wt. % silver ions, calculated as Ag²⁺.

A further preferred anti-microbial compound according to the invention, which can be used in the products according to the invention in addition to or instead of benzoates and/or parabens and/or Cu salts and/or Ag salts, is triclosan.

Triclosan (2,4,4′-trichloro-2′-hydroxydiphenyl ether) is preferably used in narrower quantity ranges, so that preferred oral and dental care and cleaning products according to the invention comprise—based on the weight thereof—0.05 to 0.75 wt. %, preferably 0.06 to 0.5 wt. %, particularly preferably 0.07 to 0.4 wt. %, exceptionally preferably 0.08 to 0.35 wt. %, and in particular 0.1 to 0.3 wt. % triclosan.

A further preferred anti-microbial compound according to the invention, which can be used in the products according to the invention in addition to or instead of benzoates and/or parabens and/or Cu salts and/or Ag salts and/or triclosan, is hexetidine.

Hexetidine, also referred to as 5-amino-1,3-bis(2-ethylhexal)hexahydro-5-methylpyrimidine or 1,3-bis(2-ethylhexyl)-hexahydro-5-methylpyrimidine-5-amine, is an antiseptic or disinfectant, which is reflected by the following formula:

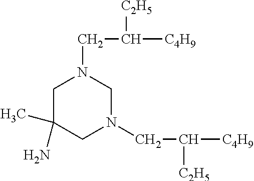

Hexetidine is likewise preferably used in the products according to the invention in narrower quantity ranges, so that preferred oral and dental care and cleaning products according to the invention comprise—based on the weight thereof—0.01 to 0.5 wt. %, preferably 0.02 to 0.4 wt. %, particularly preferably 0.03 to 0.3 wt. %, exceptionally preferably 0.04 to 0.2 wt. %, and in particular 0.05 to 0.1 wt. % 1,3-bis(2-ethylhexyl)-hexahydro-5-methylpyrimidine-5-amine (hexetidine).

The oral and dental care and cleaning products according to the invention comprise further ingredients. The use of what are known as humectants is preferred, which prevent toothpastes from drying out. In what are known as liquid dentifrices having a flow-able rheology, they serve as a matrix and are used in higher quantities. In mouthwashes and oral rinses, these alcohols are used as consistency regulators and additional sweeteners.

Preferred oral and dental care and cleaning products according to the invention here are those that comprise—based on the weight thereof—0.5 to 60 wt. %, preferably 0.75 to 55 wt. %, particularly preferably 1 to 50 wt. %, and in particular 2 to 40 wt. % of at least one polyhydric alcohol from the group sorbitol and/or glycerol and/or 1,2-propylene glycol-% or the mixtures thereof.

For certain fields of application, it may be advantageous to use only one of the three ingredients described above. Sorbitol is preferred in the majority of cases. However, mixtures of two of the three substances, or of all three substances, may be preferred in other fields of application. A mixture of glycerol, sorbitol and 1,2-propylene glyocol at a weight ratio of 1:(0.5-1):(0.1-0.5) has proven to be particularly advantageous.

In addition to sorbitol or glycerol or 1,2-propylene glycol, further suitable polyhydric alcohols are those having at least 2 OH groups, preferably mannitol, xylitol, polyethylene glycol, polypropylene glycol and the mixtures thereof. Among these compounds, those having 2 to 12 OH groups, and in particular those having 2, 3, 4, 5, 6 or 10 OH groups are preferred.

For example, polyhydroxy compounds having 2 OH groups are glycol (CH₂(OH)CH₂OH) and other 1,2-diols such as H—(CH₂)ₙ—CH(OH)CH₂OH where n=2, 3, 4, 5, 6, 7, 8, 9, 10, 11, 12, 13, 14, 15, 16, 17, 18 19, 20. According to the invention, it is also possible to use 1,3-diols such as H—(CH₂)ₙ—CH(OH)CH₂CH₂OH where n=1, 2, 3, 4, 5, 6, 7, 8, 9, 10, 11, 12, 13, 14, 15, 16, 17, 18, 19, 20. The (n,n+1)- or (n,n+2)-diols having non-terminal OH groups can likewise be used. Important representatives of polyhydroxy compounds having 2 OH groups are also the polyethylene and polypropylene glycols. Preferred further polyhydric alcohols that can be used are, for example, xylitol, propylene glycols, polyethylene glycols, in particular those having average molecular weights of 200 to 800.

The use of sorbitol is particularly preferred, so that products that, except for sorbitol, do not comprise any other polyhydric alcohols are particularly preferred.

Oral and dental care and cleaning products can moreover particularly preferably include anti-caries active substances. These may be selected from organic or inorganic fluorides, for example, such as from sodium fluoride, potassium fluoride, sodium monofluorophosphate and sodium fluorosilicate. Zinc fluoride and tin(II) fluoride are also preferred. Preferably a quantity of 0.01 to 0.2 wt. % fluorine in the form of the described compounds should be present.

Oral and dental care and cleaning products according to the invention that additionally include anti-caries active substances, preferably fluorine compound(s), in particular sodium fluoride, potassium fluoride, sodium monofluorophosphate, zinc fluoride, tin fluoride and sodium fluorosilicate, preferably in quantities of 0.01 to 5 wt. %, preferably 0.02 to 2.5 wt. %, and in particular 0.04 to 1.1 wt. %, in each case based on the total product, are preferred according to the invention.

The oral and dental care products, in particular the toothpastes, may also include substances that help desensitize the teeth, for example potassium salts such as potassium nitrate, potassium citrate, potassium chloride, potassium bicarbonate and potassium oxalate. Preferred oral and dental care and cleaning products according to the invention are characterized by comprising tooth desensitizing substances, preferably potassium salts, particularly preferably potassium nitrate and/or potassium citrate and/or potassium chloride and/or potassium bicarbonate and/or potassium oxalate, preferably in quantities of 0.5 to 20 wt. %, particularly preferably 1.0 to 15 wt. %, more preferably 1.5 to 5 wt. %, and in particular 1.75 to 2.5 wt. %, in each case based on the total product.

The products according to the invention may additionally include further wound healing and anti-inflammatory substances, such as active substances against gingivitis. Such substances may be selected from allantoin, azulene, chamomile extracts, tocopherol, panthenol, bisabolol and sage extracts, for example.

The use of abrasives is likewise preferred. Abrasives are amorphous, predominantly inorganic, largely water-insoluble, extremely fine powders that have no sharp edges. In dental and oral care products, they favor the cleaning of the teeth, while polishing the tooth surface (polishing agents).

In principle, all friction bodies known for toothpastes are suitable polishing agents, in particular those that do not include any calcium ions. Preferred suitable polishing agent components may therefore be silicas, aluminum hydroxide, aluminum oxide, sodium aluminosilicates, organic polymers or mixtures of such friction bodies.

Calcium-containing polishing components such as chalk, calcium pyrophosphate, dicalcium phosphate dihydrate, however, may be present in quantities of up to 5 wt. %, based on the total composition.

The total content of polishing agents in the dental care product preferably ranges from 5 to 50 wt. %.

Toothpastes and liquid dentifrice products that include silicas as the polishing agents are particularly preferred. Suitable silicas are silica gels, silica hydrogels and precipitated silicas. Silica gels are produced by reacting sodium silicate solutions with strong, acid mineral acids forming a hydrosol, aging to obtain the hydrogel, washing, and drying. If drying takes place under gentle conditions to water contents of 15 to 35 wt. %, what are known as silica hydrogels are obtained. Drying to water contents below 15 wt. % results in irreversible shrinkage of the previously loose structure of the hydrogel to form the dense structure of the so-called xerogel.

A second preferred suitable group of silica polishing agents are the precipitated silicas. These are obtained by precipitating silica from diluted alkali silicate solutions by adding strong acids under conditions at which aggregation into the sol and gel cannot occur. Suitable methods for A precipitated silica having a BET surface area of 15 to 110 m²/g, a particle size of 0.5 to 20 μm, wherein at least 80 wt. % of the primary particles should be below 5 μm, and a viscosity in a 30% glycerol-water (1:1) dispersion of 30 to 60 Pa·s (20° C.) in a quantity of 10 to 20 wt. % of the toothpaste is preferably suited. Preferably suitable precipitated silicas of this type moreover have rounded corners and edges and available under the trade name Sident®12 DS (DEGUSSA).

Other precipitated silicas of this type are Sident® 8 (DEGUSSA) and Sorbosil® AC 39 (Crosfield Chemicals). These silicas are characterized by a lower thickening action and a slightly higher mean particle size of 8 to 14 μm at a specific surface area of 40 to 75 m²/g (according to BET) and are particularly well-suited for liquid dentifrices. These should have a viscosity (25° C., shear rate D=10 s⁻¹) of 1 to 10 Pa·s.

In contrast, toothpastes that have a considerably higher viscosity of more than 100 Pa·s (25° C., D=10 s⁻¹) require a sufficiently high amount of silicas having a particle size of less than 5 μm, preferably at least 3 wt. % of a silica having a particle size of 1 to 3 μm. In addition to the described precipitated silicas, preferably even finer silicas known as thickening silicas, having a BET surface area of 150 to 250 m²/g, such as the commercial products Sipernat 22 LS or Sipernat 320® DS, are added to such toothpastes.

It is also possible, for example, for aluminum oxide in the form of weakly calcined alumina having a content of—and aluminum oxide to be present in a quantity of approximately 1 to 5 wt. % as a further polishing agent component. Such a suitable aluminum oxide is available under the trade name “Poliertonerde P10 feinst (ultrafine polishing alumina P10)” (Giulini Chemie).

Moreover all friction bodies known for toothpastes are suitable polishing agents, for example sodium aluminosilicates such as zeolite A, organic polymers such as polymethacrylate, or mixtures of these and the above-described friction bodies.

In summary, preferred oral and dental care and cleaning products according to the invention are those that additionally include abrasives, preferably silicas, aluminum hydroxide, aluminum oxide, calcium pyrophosphate, chalk, dicalcium phosphate dihydrate (CaHPO₄.2H₂O), sodium aluminosilicates, in particular zeolite A, organic polymers, in particular polymethacrylates or mixtures of these friction bodies, preferably in quantities of 1 to 30 wt. %, preferably 2.5 to 25 wt. %, and in particular 5 to 22 wt. %, in each case based on the total product.

The indicated quantities refer to the total amount of friction bodies, wherein individual friction bodies are preferably used in narrower quantity ranges. For example, preferred product according to the invention comprise 5 to 20 wt. %, preferably 8 to 21 wt. %, more preferably 9 to 20 wt. %, and in particular 11 to 19 wt. % silica(s). More preferred products according to the invention are characterized by comprising 0.25 to 2 wt. %, preferably 0.5 to 1.5 wt. %, and in particular 0.75 to 1.25 wt. % aluminum oxide.

Oral and dental care and cleaning products may also include substances that are effective against plaque and/or tartar, for example.

Substances effective against tartar can be chelating agents, for example, such as ethylenediaminetetraacetic acid and the sodium salts thereof, azacycloheptane diphosphonates, pyrophosphate salts such as the water-soluble dialkali or tetraalkali metal pyrophosphate salts, for example Na₄P₂O₇, K₄P₂O₇, Na₂K₂P₂O₇, Na₂H₂P₂O₇ and K₂H₂P₂O₇, or polyphosphate salts, which may be selected from water-soluble alkali metal tripolyphospates such as sodium tripolyphospate and potassium tripolyphosate, for example.

Preferred oral and dental care and cleaning products according to the invention are characterized by additionally comprising phosphate(s), preferably alkali metal phosphate(s), and in particular sodium tripolyphosphate, preferably in quantities of 1 to 10 wt. %, particularly preferably 2 to 8 wt. %, and in particular 3 to 7 wt. %, in each case based on the total product.

The consistency regulators (or binding agents) that are used are, for example, natural and/or synthetic water-soluble polymers such as alginates, carrageenates, tragacanth, starch and starch ether, cellulose ether such as carboxymethylcellulose (Na salt), hydroxyethyl cellulose, methylhydroxypropylcellulose, guar, acacia gum, agar-agar, xanthan gum, succinoglycan gum, locust bean gum, pectins, water-soluble carboxyvinyl polymers (such as Carbopol® types), polyvinyl alcohol, polyvinylpyrrolidone, polethylene glycols, in particular those having molecular weights of 1,500 to 1,000,000.

Further substances, which are suitable for viscosity control, are phyllosilicates, for example, such as montmorrillonite clays, colloidal thickening silicas, such as silica aerogels, fumed silicas or finely ground precipitated silicas. It is also possible to use viscosity-stabilizing additives from the group consisting of the cationic, zwitterionic or ampholytic nitrogenous surfactants, the hydroxypropyl-substituted hydrocolloids or the polyethylene glycol/polypropylene glycol copolymers having a mean molecular weight of 1000 to 5000, or a combination of the described compounds, in the toothpastes.

Surface-active substances may also be used in the products according to the invention. In toothpastes, they are used, for example, to support the cleaning action and optionally also to assist with foam development when brushing the teeth or rinsing the mouth, and to stabilize the polishing body dispersion in the carrier, and they are typically used both in oral rinse solutions and in toothpastes in a quantity of 0.1 to 5 wt. %.

Suitable surfactants are linear sodium alkyl sulfates having 12 to 18 carbon atoms in the alkyl group, for example. These substances additionally have an enzyme-inhibiting effect on the bacterial metabolism of the dental plaque. Further suitable surfactants are alkali salts, preferably sodium salts of alkyl polyglycol ether sulfate having 12 to 16 carbon atoms in the linear alkyl group and 2 to 6 glycol ether groups in the molecule, of linear alkane (C₁₂-_{C18}) sulfonate, of sulfosuccinic acid monoalkyl (C₁₂-C₁₈) esters, of sulfated fatty acid monoglycerides, sulfated fatty acid alkanolamides, sulfoacetic acid alkyl (C₁₂-C₁₆) esters, acyl sarcosines, acyl taurides and acyl isothionates, each having 8 to 18 carbon atoms in the acyl group. Zwitterionic, ampholytic and nonionic surfactants are also suited, for example oxethylates of fatty acid monoglycerides and -diglycerices, of sorbitan fatty acid esters and alkyl(oligo) gluocides and fatty acid amidoalky betaines.

The products according to the invention, in particular the toothpastes, can also comprise substances to increase the mineralizing potential, for example calcium-containing substances such as calcium chloride, calcium acetate and dicalcium phosphate dihydrate. The concentration of the calcium-containing substance depends on the solubility of the substance and the interaction with other substances present in the oral and dental care product.

In addition to the described mandatory components, the dental care products according to the invention may contain further auxiliary agents and additives which are known per se. One additive which has been long known as a toothpaste component is particularly effective in the dental care products according to the invention: calcium glycerophosphate, the calcium salt of glycerol-1-phosphoric acid or of glycerol-2-phosphoric acid or of glycerol-3-phosporic acid, the mirror-image isomer of glycerol-1-phosphoric acid, or a mixture of these acids. This compound has a remineralizing effect indental care products because it supplies both calcium ions and phosphate ions. Calcium glycerophosphate is preferably used in quantities of 0.01 to 1 wt. % in the dental care products according to the invention. Overall, the dental care products according to the invention may include typical auxiliary substances and additives in quantities of up to 10 wt. %.

The organoleptic properties of the dental care products according to the invention can be improved, for example, by adding flavoring oils and sweeteners.

The flavoring oils can be any of the natural and synthetic flavors typically used for oral and dental care products. Natural flavoring agents may be present either in the form of the natural essential oils isolated from drugs or in the form of the individual components isolated therefrom.

Suitable flavoring agents are, for example, peppermint oil, spearmint oil, eucalyptus oil, aniseed oil, fennel oil, caraway oil, menthyl acetate, cinnamaldehyde, anethol, vanillin, thymol, and mixtures of these components. Suitable sweeteners are, for example, saccharin sodium, sodium cyclamate, sucrose, lactose, maltose, and fructose.

Further typical auxiliary substances and additives for toothpastes and mouthwashes or oral rinse solutions are:
surface-active substances, preferably anionic, zwitterionic, amphoteric, nonionc surfactants, or a combination of several different surfactants
solvents and solubilizers, such as lower monohydric or polyhydric alcohols or ethers, such as ethanol, 1,2-propylene glycol, diethylene glycol or butyl diglycol
pigments, such as titanium dioxide
dyes
buffer substances, such as primary, secondary or tertiary alkali phosphates or citric acid/Na citrate
other wound healing or anti-inflammatory substances, such as allantoin, urea, azulene, active chamomile substances, acetylsalicylic acid derivatives or thiocyanate
further vitamins such as ascorbic acid, biotin, tocopherol or rutin
mineral salts such as manganese salts, zinc salts or magnesium salts.

It has been shown that the performance of the oral and dental care and cleaning products according to the invention can be further enhanced when the products include salivation-promoting substances. In particular, the antibacterial action, and along with it the anti-caries action and the action against gingivitis and/or periodontitis, are thereby intensified.

Salivation shall be understood to mean saliva production and secretion, and in the broader sense also in a non-physiologically increased quantity. Substances that stimulate salivation and increase the amount and/or secretion of saliva can come from a wide variety of substance classes.

A substance that is suited according to the invention, for example, is pilocarpine, which may be present in the oral and dental care and cleaning products according to the invention.

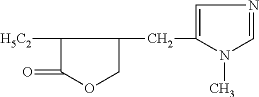

### Pilocarpine

Further salivation-promoting substances are in particular so-called pungent substances, which is to say pungent tasting and/or warmth-inducing substances. Preferred oral and dental care and cleaning products according to the invention are characterized by comprising at least one pungent tasting and/or warmth-inducing substance as the salivation-promoting substance.

The products according to the invention in this embodiment comprise a pungent tasting and/or warmth-inducing substance as the salivation-promoting ingredient. These substances induce a spicy, tingling, mouth-watering or warmth-producing effect in the user, which is to say they create an impression of warmth or a burning, prickling, beading, tickling or invigorating sensation and thereby promote salivation.

Preferred products of this embodiment according to the invention comprise the pungent tasting and/or warmth-inducing substance(s) in quantities of 0.00001 to 5 wt. %, preferably 0.0005 to 2.5 wt. %, more preferably 0.001 to 1 wt. %, particularly preferably 0.005 to 0.75 wt. %, and in particular 0.01 to 0.5 wt. %, in each case based on the weight of the total product.

A number of substances can be used as the pungent tasting or warmth-inducing substance. Preferred are in particular N-alkyl-substituted amides of unsaturated carboxylic acids, for example
2E,4E-decadienoic acid-N-methylamide
2E,4E-decadienoic acid-N-ethylamide
2E,4E-decadienoic acid-N-n-propylamide
2E,4E-decadienoic acid-N-isopropylamide
2E,4E-decadienoic acid-N-n-butylamide
2E,4E-decadienoic acid-N-(1-methylpropyl)amide
2E,4E-decadienoic acid-N-isobutylamide
2E,4E-decadienoic acid-N-tert-butylamide
2E,4Z-decadienoic acid-N-methylamide
2E,4Z-decadienoic acid-N-ethylamide
2E,4Z-decadienoic acid-N-n-propylamide
2E, 4Z-decadienoic acid-N-isopropylamide
2E, 4Z-decadienoic acid-N-n-butylamide
2E, 4Z-decadienoic acid-N-(1-methylpropyl)amide
2E, 4Z-decadienoic acid-N-isobutylamide
2E,4Z-decadienoic acid-N-tert-butylamide
2E,4E,8Z-decatrienoic acid-N-methylamide
2E,4E,8Z-decatrienoic acid-N-ethylamide
2E, 4E, 8Z-decatrienoic acid-N-n-propylamide
2E,4E,8Z-decatrienoic acid-N-isopropylamide
2E,4E,8Z-decatrienoic acid-N-n-butylamide
2E, 4E, 8Z-decatrienoic acid-N-(1-methylpropyl)amide
2E, 4E, 8Z-decatrienoic acid-N-isobutylamide
2E, 4E, 8Z-decatrienoic acid-N-tert-butylamide
2E,4Z,8Z-decatrienoic acid-N-methylamide
2E, 4Z, 8Z-decatrienoic acid-N-ethylamide
2E, 4Z, 8Z-decatrienoic acid-N-n-propylamide
2E,4Z,8Z-decatrienoic acid-N-isopropylamide
2E,4Z,8Z-decatrienoic acid-N-n-butylamide
2E, 4Z, 8Z-decatrienoic acid-N-(1-methylpropyl)amide
2E, 4Z, 8Z-decatrienoic acid-N-isobutylamide
2E, 4Z, 8Z-decatrienoic acid-N-tert-butylamide
2E,4E,8E-decatrienoic acid-N-methylamide
2E, 4E, 8E-decatrienoic acid-N-ethylamide
2E, 4E, 8E-decatrienoic acid-N-n-propylamide
2E,4E,8E-decatrienoic acid-N-isopropylamide
2E,4E,8E-decatrienoic acid-N-n-butylamide
2E, 4E, 8E-decatrienoic acid-N-(1-methylpropyl)amide
2E, 4E, 8E-decatrienoic acid-N-isobutylamide
2E, 4E, 8E-decatrienoic acid-N-tert-butylamide
2E,4Z,8E-decatrienoic acid-N-methylamide
2E,4Z,8E-decatrienoic acid-N-ethylamide
2E,4Z,8E-decatrienoic acid-N-n-propylamide
2E,4Z,8E-decatrienoic acid-N-isopropylamide
2E,4Z,8E-decatrienoic acid-N-n-butylamide
2E, 4Z, 8E-decatrienoic acid-N-(1-methylpropyl)amide
2E, 4Z, 8E-decatrienoic acid-N-isobutylamide
2E,4Z,8E-decatrienoic acid-N-tert-butylamide
2E,6Z,8E-decatrienoic acid-N-methylamide
2E,6Z,8E-decatrienoic acid-N-ethylamide
2E,6Z,8E-decatrienoic acid-N-n-propylamide
2E,6Z,8E-decatrienoic acid-N-isopropylamide
2E,6Z,8E-decatrienoic acid-N-n-butylamide
2E, 6Z, 8E-decatrienoic acid-N-(1-methylpropyl)amide
2E, 6Z, 8E-decatrienoic acid-N-isobutylamide
2E,6Z,8E-decatrienoic acid-N-tert-butylamide
2E,6E,8E-decatrienoic acid-N-methylamide
2E,6E,8E-decatrienoic acid-N-ethylamide
2E,6E,8E-decatrienoic acid-N-n-propylamide
2E,6E,8E-decatrienoic acid-N-isopropylamide
2E,6E,8E-decatrienoic acid-N-n-butylamide
2E, 6E, 8E-decatrienoic acid-N-(1-methylpropyl)amide
2E, 6E, 8E-decatrienoic acid-N-isobutylamide
2E,6E,8E-decatrienoic acid-N-tert-butylamide
2E,6Z,8Z-decatrienoic acid-N-methylamide
2E,6Z,8Z-decatrienoic acid-N-ethylamide
2E,6Z,8Z-decatrienoic acid-N-n-propylamide
2E,6Z,8Z-decatrienoic acid-N-isopropylamide
2E,6Z,8Z-decatrienoic acid-N-n-butylamide
2E, 6Z, 8Z-decatrienoic acid-N-(1-methylpropyl)amide
2E, 6Z, 8Z-decatrienoic acid-N-isobutylamide
2E,6Z,8Z-decatrienoic acid-N-tert-butylamide
2E,6E,8Z-decatrienoic acid-N-methylamide
2E, 6E, 8Z-decatrienoic acid-N-ethylamide
2E, 6E, 8Z-decatrienoic acid-N-n-propylamide
2E,6E,8Z-decatrienoic acid-N-isopropylamide
2E, 6E, 8Z-decatrienoic acid-N-n-butylamide
2E, 6E, 8Z-decatrienoic acid-N-(1-methylpropyl)amide
2E, 6E, 8Z-decatrienoic acid-N-isobutylamide
2E,6E,8Z-decatrienoic acid-N-tert-butylamide
2E,7Z,9E-undecatrienoic acid-N-methylamide
2E,7Z,9E-undecatrienoic acid-N-ethylamide
2E, 7Z,9E-undecatrienoic acid-N-n-propylamide
2E,7Z,9E-undecatrienoic acid-N-isopropylamide
2E, 7Z,9E-undecatrienoic acid-N-n-butylamide
2E,7Z,9E-undecatrienoic acid-N-(1-methylpropyl)amide
2E, 7Z,9E-undecatrienoic acid-N-isobutylamide
2E, 7Z,9E-undecatrienoic acid-N-tert-butylamide
2E,7E,9E-undecatrienoic acid-N-methylamide
2E,7E,9E-undecatrienoic acid-N-ethylamide
2E, 7E,9E-undecatrienoic acid-N-n-propylamide
2E,7E,9E-undecatrienoic acid-N-isopropylamide
2E, 7E,9E-undecatrienoic acid-N-n-butylamide
2E,7E,9E-undecatrienoic acid-N-(1-methylpropyl)amide
2E, 7E,9E-undecatrienoic acid-N-isobutylamide
2E, 7E,9E-undecatrienoic acid-N-tert-butylamide
2E,7Z,9Z-undecatrienoic acid-N-methylamide
2E,7Z,9Z-undecatrienoic acid-N-ethylamide
2E, 7Z,9Z-undecatrienoic acid-N-n-propylamide
2E,7Z,9Z-undecatrienoic acid-N-isopropylamide
2E, 7Z,9Z-undecatrienoic acid-N-n-butylamide
2E,7Z,9Z-undecatrienoic acid-N-(1-methylpropyl)amide
2E, 7Z,9Z-undecatrienoic acid-N-isobutylamide
2E,7Z,9Z-undecatrienoic acid-N-tert-butylamide
2E,7Z,9E-undecatrienoic acid-N-methylamide
2E,7Z,9E-undecatrienoic acid-N-ethylamide
2E,7Z,9E-undecatrienoic acid-N-n-propylamide
2E,7Z,9E-undecatrienoic acid-N-isopropylamide
2E,7Z,9E-undecatrienoic acid-N-n-butylamide
2E,7Z,9E-undecatrienoic acid-N-(1-methylpropyl)amide
2E,7Z,9E-undecatrienoic acid-N-isobutylamide
2E,7Z,9E-undecatrienoic acid-N-tert-butylamide

It goes without saying that other substitution patterns at the nitrogen atom are also possible and preferred, for example longer-chained n-alkyl groups ( . . . —N-n-pentylamide, . . . —N-n-pentylamide, . . . —N-n-pentylamide, . . . —N-n-pentylamide, . . . —N-n-pentylamide, . . . —N-n-hexylamide, . . . —N-n-heptylamide, . . . —N-n-octylamide, . . . —N-n-nonylamide, . . . —N-n-decylamide, . . . —N-n-undecylamide, . . . —N-n-dodecylamide, . . . —N-n-tridecylamide, and so forth) or disubstituted . . . —N,N-dialkylamides such as . . . —N,N-dimethylamide, . . . —N,N-diethylamide, . . . —N,N-di-n-propylamide, . . . —N,N-diisopropylamide, . . . —N,N-di-n-butylamide, . . . —N,N-di(1-methylpropyl)amide, . . . —N,N-diisobutylamide, . . . —N,N-di-tert-butylamide, . . . —N,N-methyl-ethylamide, . . . —N,N-methyl-n-propylamide, . . . —N,N-methyl-isopropylamide, . . . —N,N-ethyl-n-propylamide, . . . —N,N-ethyl-isopropylamide, and so forth.

Several of the described compounds are particularly preferred within the scope of the present invention. These are listed below:

2E,6Z,8E-decatrienoic acid-N-isobutylamide (N-isobutyl-2E,6Z,8E-decatrienamide, also referred to as Spilanthol or Affinin):

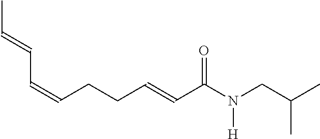 [SMILES: C/C=C/C=C\CC/C=C/C(=O)NCC(C)C]

2E,4E,8Z-decatrienoic acid-N-isobutylamide (N-isobutyl-2E,4E,8Z-decatrienamide, also referred to as Isoaffinin):

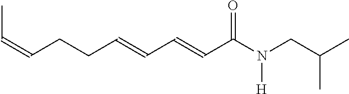 [SMILES: C/C=C\CC/C=C/C=C/C(=O)NCC(C)C]

2E,7Z,9E-undecatrienoic acid-N-isobutylamide (N-isobutyl-2E,7Z,9E-undecatrienamide):

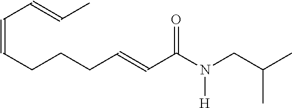 [SMILES: C/C=C/C=C\CCC/C=C/C(=O)NCC(C)C]

2E,4Z-decadienoic acid-N-isobutylamide (cis-pellitorin):

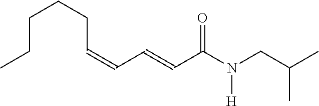 [SMILES: CCCCC/C=C\C=C\C(=O)NCC(C)C]

2E,4E-decadienoic acid-N-isobutylamide (trans-pellitorin):

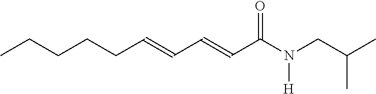 [SMILES: CCCCC/C=C/C=C/C(=O)NCC(C)C]

ferulic acid amide, for example
ferulic acid-N-vanillylamide:

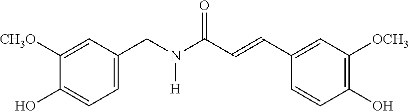

N-[2-(4-hydroxy-3-methoxyphenyl)ethyl]-3-(4-hydroxy-3-methoxy-phenyl)-(2E)-propenoic acid amide (trans-feruloylmethoxytyramine):

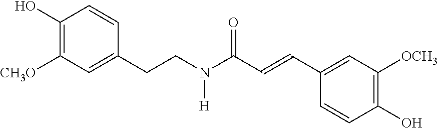

N-[2-(4-hydroxy-3-methoxyphenyl)ethyl]-3-(4-hydroxy-3-methoxy-phenyl)-(2Z)-propenoic acid amide (cis-feruloylmethoxytyramine):

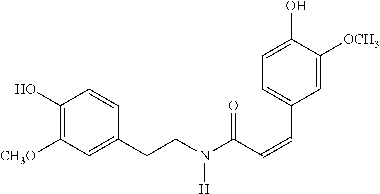

N-[2-(4-hydroxy-3-methoxyphenyl)ethyl]-3-(4-hydroxy-3-methoxy-phenyl)-propanoic acid amide (dihydroferuloylmethoxytyramine):

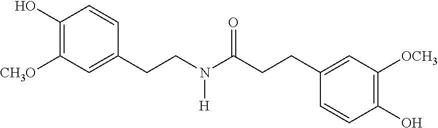

N-[2-(3,4-dihydroxyphenyl)ethyl]-3-(4-hydroxy-3-methoxy-phenyl)-(2E)-propenoic acid amide(trans-feruloyldopamine)

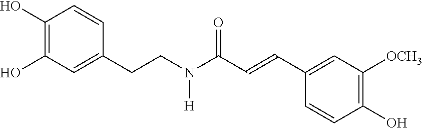 [SMILES: COc1cc(/C=C/C(=O)NCCc2ccc(O)c(O)c2)ccc1O]

N-[2-(3,4-dihydroxyphenyl)ethyl]-3-(4-hydroxy-3-methoxy-phenyl)-(2Z)-propenoic acid amide (cis-feruloyldopamine):

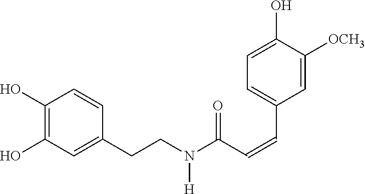 [SMILES: COc1cc(/C=C\C(=O)NCCc2ccc(O)c(O)c2)ccc1O]

N-[2-(4-hydroxyphenyl)ethyl]-3-(3,4-dihydroxyphenyl)-(2E)-propenoic acid amide (trans-caffeoyltyramine):

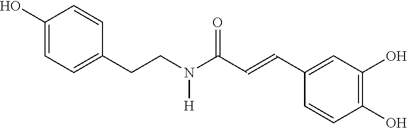 [SMILES: O=C(/C=C/c1ccc(O)c(O)c1)NCCc1ccc(O)cc1]

N-[2-(4-hydroxyphenyl)ethyl]-3-(3,4-dihydroxyphenyl)-(2Z)-propenoic acid amide (cis-caffeoyltyramine):

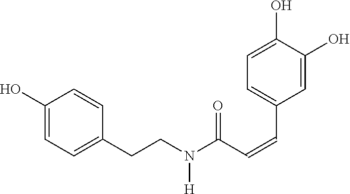 [SMILES: O=C(/C=C\c1ccc(O)c(O)c1)NCCc1ccc(O)cc1]

N-[2-(3,4-dimethoxyphenyl)ethyl]-3-(3,4-dimethoxyphenyl)-(2E)-propenoic acid amide (trans-rubenamine):

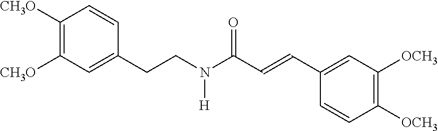

N-[2-(3,4-dimethoxyphenyl)ethyl]-3-(3,4-dimethoxy-phenyl)-(2Z)-propenoic acid amide (cis-rubenamine):

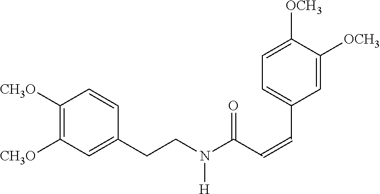

Further pungent substances that can be used particularly preferably within the scope of the present invention are natural plant extracts, for example. Plant extracts that have a pungent taste can be any physiologically safe plant extracts that induce a pungent or warm sensory impression. Preferred pungent tasting plant extracts are, for example, pepper extract (Piper ssp., in particular Piper nigrum), water pepper extract (Polygonum ssp., in particular Polygonum hydropiper), extracts of Allium ssp. (in particular onion and garlic extracts), extracts of radish (Raphanus ssp.), horseradish extracts (Cochlearia armoracia), extracts of black (Brassica nigra), wild or yellow mustard (Sinapis ssp., in particular Sinapis arvensis and Sinapis alba), pyrethrum extracts (Anacyclus ssp., in particular Anacylcus pyrethrum L.), purple coneflower extracts (Echinaceae ssp.), extracts of Szechuan pepper (Zanthoxylum ssp., in particular Zanthoxylum piperitum), spilanthes extract (Spilanthes ssp., in particular Spilanthes acmella), chili extract (Capsicum ssp., in particular Capsicum frutescens), grains of paradise extract (Aframomum ssp., in particular Aframomum melegueta [Rose] K. Schum.), ginger extract (Zingiber ssp., in particular Zingiber officinale), and galangal extract (Kaempferia galanga or Alpinia galanga).

A particularly suitable substance is gingerol obtained from ginger extract. N-ethyl-p-menthane-3-carboxamide (N-ethyl-5-methyl-2-isopropylcyclohexane carboxamide) may also be used. Other pungent or warmth-incuding substances may be, for example, capsaicin, dihydrocapsaicin, gingerol, paradol, shogaol, piperin, carboxylic acid-N-vanillylamide, in particular nonanoic acid-N-vanillylamide, 2-alkenoic acid amides, in particular 2-nonenoic acid-N-isobutylamide, 2-nonenoic acid-N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 3-hydroxy-4-methoxybenzyl alcohol, alkyl ethers of 3,4-dimethoxybenzyl alchohol, alkyl ethers of 3-ethoxy-4-hydroxybenzyl alcohol, alkyl ethers of 3,4-methylenedioxybenzyl alcohol, nicotinaldehyde, methyl nicotinate, propyl nicotinate, 2-butoxyethyl nicotinate, benzyl nicotinate, 1-acetoxychavicol, polygodial, or isodrimeninol.

Preferred remineralizing products according to the invention are characterized by comprising at least one pungent substance from the group of N-alkyl-substituted amides of unsaturated carboxylic acid, preferably
a. 2E,6Z,8E-decatrienoic acid-N-isobutylamide (Spilanthol) and/or
b. 2E,4E,8Z-decatrienoic acid-N-isobutylamide and/or
c. 2E,7Z,9E-undecatrienoic acid-N-isobutylamide and/or
d. 2E,4Z-decadienoic acid-N-isobutylamide (cis-pellitorin) and/or
e. 2E,4E-decadienoic acid-N-isobutylamide (trans-pellitorin) and/or
f. ferulic acid-N-vanillylamide and/or
g. N-[2-(4-hydroxy-3-methoxyphenyl)ethyl]-3-(4-hydroxy-3-methoxy-phenyl)-(2E)-propenoic acid amide (trans-feruloylmethoxytyramine) and/or
h. N-[2-(4-hydroxy-3-methoxyphenyl)ethyl]-3-(4-hydroxy-3-methoxy-phenyl)-(2Z)-propenoic acid amide (cis-feruloylmethoxytyramine) and/or
i. N-[2-(4-hydroxy-3-methoxyphenyl)ethyl]-3-(4-hydroxy-3-methoxy-phenyl)-propanoic acid amide (dihydroferuloylmethoxytyramine) and/or
j. N-[2-(3,4-dihydroxyphenyl)ethyl]-3-(4-hydroxy-3-methoxy-phenyl)-(2E)-propenoic acid amide (trans-feruloyldopamine) and/or
k. N-[2-(3,4-dihydroxyphenyl)ethyl]-3-(4-hydroxy-3-methoxy-phenyl)-(2Z)-propenoic acid amide (cis-feruloyldopamine) and/or
l. N-[2-(4-hydroxyphenyl)ethyl]-3-(3,4-dihydroxyphenyl)-(2E)-propenoic acid amide (trans-caffeoyltyramine) and/or
m. N-[2-(4-hydroxyphenyl)ethyl]-3-(3,4-dihydroxyphenyl)-(2Z)-propenoic acid amide (cis-caffeoyltyramine) and/or
n. N-[2-(3,4-dimethoxyphenyl)ethyl]-3-(3,4-dimethoxyphenyl)-(2E)-propenoic acid amide (trans-rubenamine) and/or
o. N-[2-(3,4-dimethoxyphenyl)ethyl]-3-(3,4-dimethoxy-phenyl)-(2Z)-propenoic acid amide (cis-rubenamine)

In addition to the described pungent substances or instead of the same, it is also possible to incorporate further pungent tasting and/or warmth-inducing substances into the products according to the invention.

Within the scope of the present invention, alkyl-substituted dioxanes of formula

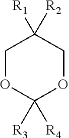

have proven to be particularly suitable, where R1 and R2, independently of each other, are selected from —H, —CH₃, —CH₂CH₃, and R3 and R4, independently of each other, are selected from —H, —CH₃, —CH₂CH₃, —CH₂CH₂CH₃, —CH(CH₃)₂.

Within the scope of the present invention, phenyl esters of formula

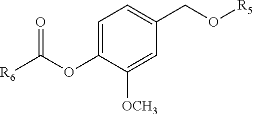

have moreover proven to be particularly suitable, where R5 denotes —CH₃ or a straight-chain or branched alkyl or alkenyl group have 2 to 8 carbon atoms, and R6 denotes —CH₃ or a straight-chain or branched alky or alkenyl group having 2 to 8 carbon atoms or an alkoxy group having 1 to 3 carbon atoms.

Within the scope of the present invention, carvone acetals of formula

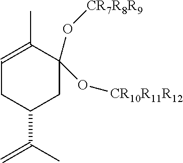

have moreover proven to be particularly suitable, where R7 to R12, independently of each other, are selected from —H, —CH₃, —CH₂CH₃, —CH₂CH₂CH₃, —CH(CH₃)₂, —CH₂ CH₂CH₂CH₃, —CH₂CH(CH₃)₂, —CH(CH₃)CH₂CH₃, —C(CH₃)₃, or R9 and R10 together denote a chemical bond or a group —(CR13R14)ₓ, where x denotes the values 1 or 2, and R13 and R14, independently of each other, are selected from —H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃.

Products according to the invention can be formulated as toothpastes or cream dentifrices. A further subject matter of the present invention is a method for preventing and treating caries and/or for combatting halitosis and/or for treating gingivitis or periodontitis, in which a preparation according to the invention is applied to a toothbrush and used to brush the teeth.

The compositions according to the invention can also be formulated as oral rinse solutions or mouthwashes. A further subject matter of the present invention is a method for preventing and treating caries and/or combating halitosis and/or for treating gingivitis or periodontitis, in which a preparation according to the invention in the form of an oral rinse solution is introduced into the oral cavity, where it is left for a period of at least 10 seconds, preferably at least 20 seconds, and in particular at least 45 seconds.

The action of antibacterial substances can be synergistically enhanced by using the polylactic acid particles. A further subject matter of the present invention is thus the use of polylactic acid particles to enhance the action of antibacterial compounds. What was said above with respect to the products according to the invention applies, mutatis mutandis, with respect to preferred embodiments of the uses according to the invention and the methods according to the invention.

### Examples

Oral rinse solutions (all information in wt. %):

| | M1 | M2 | M3 | M4 | M5 | M6 | M7 | M8 |
|---|---|---|---|---|---|---|---|---|
| Sorbitol 70% | 3.0 | 3.0 | 3.0 | 3.0 | 3.0 | 3.0 | 3.0 | 3.0 |
| Polylactic acid | 0.1 | 1.0 | 2.5 | 5.0 | 7.5 | 10.0 | 12.5 | 15.0 |
| Methyl paraben | 0.2 | | | | | | | |
| Ethyl paraben | | 0.2 | | | 0.1 | | | |
| Propyl paraben | | | 0.2 | | 0.1 | | | |
| Butyl paraben | | | | 0.2 | | | | |
| Silver citrate | | | | | | 4 ppm | | |
| | | | | | | Ag⁺ | | |
| Copper(II) sulfate | | | | | | | 5 ppm | |
| | | | | | | | Cu⁺⁺ | |
| Triclosan | | | | | | | | 0.4 |
| Sodium fluoride | 0.05 | 0.05 | 0.05 | 0.05 | 0.05 | 0.045 | 0.045 | 0.045 |
| Saccharin sodium | 0.03 | 0.03 | 0.03 | 0.03 | 0.03 | 0.03 | 0.03 | 0.03 |
| PEG-60 | 0.86 | 0.86 | 0.86 | 0.86 | 0.86 | 0.86 | 0.86 | 0.86 |
| hydrogenated | | | | | | | | |
| Flavoring agent | 0.1 | 0.1 | 0.1 | 0.1 | 0.1 | 0.1 | 0.1 | 0.1 |
| Water, deionized | up to | up to | up to | up to | up to | up to | up to 100 | up to 100 |
| | 100 | 100 | 100 | 100 | 100 | 100 | | |

(1): Ecoscrub ® 50 PC, maximum particle size 297 μm

Toothpastes having the following composition (all information in wt. %) were produced:

| | E1 | E2 | E3 | E4 | E5 |
|---|---|---|---|---|---|
| Sorbit (70% DAB) | 60 | 62.5 | 65 | 67.5 | 70.0 |
| Ethanol (96%) | — | | — | — | 2.0 |
| Precipitated silica: | 10.0 | 10.0 | 10.0 | 10.0 | 12.0 |
| Sident 8 | | | | | |
| Precipitated silica: | 8.5 | 8.5 | 8.5 | 8.5 | — |
| Sident 22S | | | | | |
| Ultrafine polishing | — | 1.0 | — | — | — |
| alumina P10 | | | | | |
| Disodium phosphate, | 0.1 | 0.1 | 0.1 | 0.1 | 0.1 |
| anhydrous | | | | | |
| Trisodium phosphate, | 0.2 | 0.2 | 0.2 | 0.2 | — |
| anhydrous | | | | | |
| Na monofluorophosphate | — | | 1.0 | 1.0 | 1.0 |
| Na2PO3F | | | | | |
| Sodium fluoride | 0.32 | 0.32 | — | — | — |
| Polyethylene glycol | 1.5 | 1.5 | 1.5 | 1.5 | 1.5 |
| (MG: 1500) | | | | | |
| Titanium dioxide | 0.5 | 0.5 | 0.5 | 0.5 | — |
| Na lauryl sulfate | 1.5 | 1.5 | 1.5 | 1.5 | 1.5 |
| Saccharin No | 0.1 | 0.1 | 0.1 | 0.1 | 0.1 |
| Xanthan | 0.4 | 0.4 | 0.4 | 0.4 | 0.4 |
| Flavoring agent | 1.2 | 1.2 | 1.2 | 1.2 | 1.2 |
| Polylactic acid | 0.1 | 0.5 | 1.0 | 1.5 | 2.5 |
| particles (1) | | | | | |
| Ethyl paraben | 0.3 | | | | |
| Methyl paraben | | 0.3 | | | |
| Silver citrate | | | 4 ppm | | |
| | | | Ag⁺ | | |
| Copper(II) sulfate | | | | 5 ppm | |
| | | | | Cu⁺⁺ | |
| Zinc sulfate | | | | | 0.3 |
| heptahydrate | | | | | |
| Water, deionized | up to | up to | up to | up to | up to |
| | 100 | 100 | 100 | 100 | 100 |

(1): Ecoscrub ® 50 PC, maximum particle size 297 μm

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oral and dental care and cleaning product, comprising, based on the weight thereof,
a) 0.001 to 25 wt. % polylactic acid particles, and
b) 0.00001 to 5 wt. % of at least one antibacterial compound from the groups consisting
a. of the benzoates and/or
b. of the parabens and/or
c. of the Cu salts and/or
d. of the Ag salts and/or
e. triclosan and/or
f. hexetidine.

2. The oral and dental care and cleaning product according to claim 1, wherein the product comprises, based on the weight thereof, 0.002 to 20 wt. % of polylactic acid particles.

3. The oral and dental care and cleaning product according to claim 1, wherein the product comprises, based on the weight thereof, 0.004 to 15 wt. % polylactic acid particles.

4. The oral and dental care and cleaning product according to claim 1, wherein the product comprises, based on the weight thereof, 0.005 to 12.5 wt. % polylactic acid particles.

5. The oral and dental care and cleaning product according to claim 1, wherein the polylactic acid particles have particle sizes of 1 to 1000 μm.

6. The oral and dental care and cleaning product according to claim 1, wherein the polylactic acid particles have particle sizes of 2 to 750 μm.

7. The oral and dental care and cleaning product according to claim 1, wherein the polylactic acid particles have particle sizes of 10 to 500 μm.

8. The oral and dental care and cleaning product according to claim 1, wherein the polylactic acid particles have mean ECD values of 10 to 1000 μm.

9. The oral and dental care and cleaning product according to claim 1, wherein the polylactic acid particles have mean ECD values of 50 to 500 μm.

10. The oral and dental care and cleaning product according to claim 1, wherein the polylactic acid particles have a mean solidity of 0.4 to 0.9

11. The oral and dental care and cleaning product according to claim 1, wherein the polylactic acid particles have a mean solidity of 0.5 to 0.8.

12. The oral and dental care and cleaning product according to claim 1, wherein the polylactic acid particles have a mean circularity C of 0.1 to 0.6.

13. The oral and dental care and cleaning product according to claim 1, wherein the antibacterial compound includes 0.01 to 4 wt. % of p-hydroxybenzoic acid methy-, ethyl-, or propyl ester or mixtures thereof.

14. The oral and dental care and cleaning product according to claim 1, wherein the antibacterial compound includes 0.1 to 1.0 wt. % p-hydroxybenzoic acid methyl-, -ethyl- or -propyl ester or the mixtures thereof.

15. The oral and dental care and cleaning product according to claim 1, wherein the antibacterial compound includes 0.01 to 4 wt. % parabens from the group methyl paraben, ethyl paraben and mixtures thereof.

16. The oral and dental care and cleaning product according to claim 1, wherein the antibacterial compound include 0.1 to 1.0 wt. % parabens from the group methyl paraben, ethyl paraben or the mixtures thereof.

17. The oral and dental care and cleaning product according to claim 1, wherein the antibacterial compound includes 0.00001 to 0.1 wt. % silver ions, calculated as Ag⁺.

18. The oral and dental care and cleaning product according to claim 1, wherein the antibacterial compound includes 0.00001 to 0.1 wt. % copper ions, calculated as Cu²⁺.

19. The oral and dental care and cleaning product according to claim 1, wherein the antibacterial compound includes 0.05 to 0.75 wt. % triclosan.

20. The oral and dental care and cleaning product according to claim 1, wherein the antibacterial compound includes 0.01 to 0.5 wt. % hexetidine.
